(19) United States
(12) Patent Application Publication
(10) Pub. No.: US 2005/0148031 A1
(43) Pub. Date: Jul. 7, 2005

(54) Catalytic efficiency and/or specificity of non-native substrates of enzymes

(76) Inventors: Nancy L. Allbritton, Irvine, CA (US); Lee Bardwell, Irvine, CA (US); Christopher E. Sims, Irvine, CA (US)

Correspondence Address: SONNENSCHEIN NATH & ROSENTHAL LLP, 8000 Sears Tower, Chicago, IL, 60606, US

(21) Appl. No.: 10/754,438

(22) Filed: Jan. 9, 2004

Related U.S. Application Data
(60) Provisional application No. 60/439,277, filed on Jan. 10, 2003.

(51) Int. Cl. G01N 33/53; G01N 33/567; C07K 16/40; C07K 14/47

(52) U.S. Cl. 435/7.2; 530/388.26; 530/409

(57) ABSTRACT

A method of introducing a fluorescent label into a cell, comprising: exposing a reporter to the cell, wherein the reporter comprises: a peptide substrate for an enzyme, a docking domain for the enzyme, attached to the peptide substrate, a membrane traversing moiety, and the label.

### RELATED APPLICATIONS

This application claims priority to U.S. Provisional Application Ser. No. 60/439,277, filed Jan. 10, 2003, which is hereby incorporated by reference.

### STATEMENT OF ACKNOWLEDGMENT OF GOVERNMENT SUPPORT

This invention was made with Government support under Grant Nos. GM60366, CA29170, GM29539 and NS29562 awarded by the National Institutes of Health. The Government has certain rights in this invention.

### BACKGROUND

Eukaryotic cells respond to developmental cues, regulatory signals and environmental stimuli with diverse changes in gene expression. A network of protein kinases orchestrates many of these responses. Precise regulation of protein kinase networks is critical in order for cells to respond appropriately to signals in their environment. Misregulation of these kinase networks corresponds to a variety of human diseases, such as cancer. Thus, understanding the regulation of protein kinase networks is important to understanding normal cellular physiology and to developing new tools for treating human diseases that result from abnormal cellular physiology.

Understanding the regulation of these networks is complicated by the diverse range of signals and responses that use the same protein kinase pathways and by the high degree of sequence similarity between many of the protein kinases involved. One mechanism that influences efficiency, as well as specificity, is the interaction of protein kinases through high-affinity protein-protein docking domains (Bardwell & Thorner, 1996; Holland & Cooper, 1999; Sharrocks et al., 2000; Enslen & Davis, 2001). These interactions are distinct from the transient interactions that occur between the active site of a protein kinase and the phosphoacceptor site in its substrate. In contrast to substrate phosphoacceptor site-active site interactions, binding of a kinase to a substrate containing a docking domain results in an increase in the local concentration of the substrate around the enzyme, thereby promoting an increase in the efficiency of substrate phosphorylation (Sharrocks et al., 2000). For example, each of the three major sub-families of mitogen-activated protein kinases (MAPK) has been shown to bind transcription factors containing docking domains, and in each case, the docking domain serves to enhance substrate phosphorylation.

Docking domains also impart specificity for protein kinase recognition of their substrates (Sharrocks et al., 2000). For example, c-Jun is regulated by members of the JNK sub-family of MAP kinases, although differences in the c-Jun-binding activity of different isoforms of this sub-family have been reported. Likewise, the docking domains found in other transcription factors, MEF2A and MEF2C, only direct their phosphorylation by a subset of kinase isoforms that belong to the p38 sub-family of MAP kinases. Yet other docking domains are recognized-by two different enzymes of MAP kinase family. For example, the docking domain in Elk-1 directs its phosphorylation by both ERK and JNK sub-families of MAP kinases.

These domains are typically organized in a similar fashion, although no clear overall consensus domain has emerged from an analysis of known and putative kinase docking domains. For example, a L×L motif located 3-5 amino acids C-terminal to a stretch of basic amino acid residues is a typical arrangement for docking domains of the MAP kinase family (e.g., (R/K)₂-(X)₂₋₆-L/I-X-L/I). Differences in the composition of these domains vary for substrates that are recognized by different sub-families of MAP kinases (Sharrocks et al., 2000). For example, p38 kinase substrates like MEF2C have a triplet of hydrophobic amino acid residues that replaces the L×L motif. Other substrates have different docking domains in the form of an additional hydrophobic amino acid residue that lies N-terminal to the stretch of basic amino acid residues.

A comparison of different protein kinase substrates and interaction partners has revealed different spatial arrangements of the docking domains relative to their phosphoacceptor sites (Sharrocks et al., 2000). For example, the docking domain lies N-terminal to the phosphoacceptor site found in Elk-1 and MEF2A. For other protein substrates, such as Rsk-1, the docking domain lies C-terminal to the phosphoacceptor site. Furthermore, the linear distance separating the docking domains from their respective phosphoacceptor sites can vary from 50 amino acids to greater than 150 amino acids (Sharrocks et al., 2000).

Numerous docking domains have been characterized to date. These domains include: (1) the aforementioned MAPK family of docking domains that are involved in the specific recognition of substrates for the sub-families of MAP kinases (JNK, p38, and ERK); (2) the four amino acid “FXFP” docking motif from the Ets-family of transcription factors that are recognized by the extracellular related kinase (ERK); (3) amino acid sequences of SH2 and SH3 docking domains that are recognized by tyrosine kinases such as Abl and Src and by phosphotyrosine phosphatases like Shp1 and Shp2; (4) the “FXXFDY” docking domain from PRK2 that interacts with the PDK1 enzyme; and (5) an amphipathic helix-loop-helix motif from FRAT residues 188-226 that acts like a docking domain with a site on GSK3. These and other docking domains have been reviewed in the literature (Sharrocks et al., 2000; Biondi & Nebreda, 2003; Neel et al., 2003; Pawson & Nash, 2003; Sidhu et al., 2003; Yu & Lemmon, 2003), which are hereby incorporated by reference.

Nine distinct groups of protein kinases are known: CMGC, STE, AGC, CAMK, CK1, TKL, TK, Other, and Atypical. A summary of human protein kinases that recognize docking domains in their respective protein substrates is presented in Table 1.

TABLE 1
Docking domains from the human genome¹

| | | Sub- | | |
|---|---|---|---|---|
| Group | Family | Family² | Kinase | Reference(s)³ |
| CMGC | CDK | | CDK2 | Adams et al. (1996) |
| | | | | Zheleva et al. (2002) |
| ″ | CDKL | | | |
| ″ | CKL | | | |
| ″ | DYRK | | | |
| ″ | GSK | | GSK3β | Dajani et al. (2001) |
| ″ | MAPK | ERK | ERK1, 2 | Bardwell et al. (2001) |
| | | | | Yang et al. (1998) |
| ″ | ″ | JNK | | Kallunki et al. (1996) |
| | | | | Ho et al. (2003) |
| ″ | ″ | p38 | | Enslen et al. (2000) |
| | | | | Yang et al. (1999) |
| ″ | RCK | | | |
| ″ | SRPK | | | |
| STE | STE-Unique | | | |
| ″ | STE20 | | | |
| ″ | STE11 | | | |
| ″ | STE7 | | MEK 1, 2 | Tanoue et al. (2000) |
| | | | | Rubinfeld et al. (1999) |
| ″ | ″ | | MKK4 | Tanoue et al. (2000) |
| ″ | ″ | | MKK6 | Tanoue et al. (2000) |
| CAMK | CAMKL | | | |
| ″ | CAMK1 | | | |
| ″ | CAMK2 | | | |
| ″ | CAMK- | | | |
| | Unique | | | |
| ″ | CASK | | | |
| ″ | DAPK | | | |
| ″ | DCAMKL | | | |
| ″ | MAPKAPK | | | |
| ″ | MLCK | | | |
| ″ | PHK | | | |
| ″ | PIM | | | |
| ″ | PKD | | | |
| ″ | PSK | | | |
| ″ | RAD53 | | | |
| ″ | Trbl | | | |
| ″ | Trio | | | |
| ″ | TSSK | | | |
| AGC | AKT | | | |
| ″ | DMPK | | | |
| ″ | GRK | | | |
| ″ | MAST | | | |
| ″ | NDR | | | |
| ″ | NDR | | | |
| ″ | PKB | | PDK1 | Biondi et al. (2002) |
| ″ | PKC | | | |
| ″ | PKG | | | |
| ″ | PKN | | | |
| ″ | RSK | RSK | RSK2 | Clark et al. (2001) |
| ″ | RSKL | | | |
| ″ | SGK | | | |
| ″ | YANK | | | |
| CK1 | CK1 | | | |
| ″ | TTBK | | | |
| ″ | VRK | | | |
| TKL | IRAK | | | |
| ″ | LISK | | | |
| ″ | LRRK | | | |
| ″ | MLK | | | |
| ″ | MLK | | | |
| ″ | RAF | | | |
| ″ | RIPK | | | |
| ″ | RIPK | | | |
| ″ | STKR | | | |
| ″ | TKL-Unique | | | |
| TK | Abl | | Abl | Duyster et al. (1995) |
| ″ | Ack | | | |
| ″ | Alk | | | |
| ″ | Axl | | | |
| ″ | CCK4 | | | |
| ″ | Csk | | | |
| ″ | DDR | | | |
| ″ | EGFR | | | |
| ″ | Eph | | | |
| ″ | Fak | | | |
| ″ | Fak | | | |
| ″ | Fer | | | |
| ″ | FGFR | | | |
| ″ | InsR | | | |
| ″ | JakA | | | |
| ″ | Lmr | | | |
| ″ | Met | | | |
| ″ | MUSK | | | |
| ″ | PDGFR | | | |
| ″ | Ret | | | |
| ″ | Ror | | | |
| ″ | Ryk | | | |
| ″ | Sev | | | |
| ″ | Src | | Src | Guappone & Flynn |
| | | | | (1997) |
| | | | | Mayer et al. (1995) |
| ″ | Syk | | | |
| ″ | Tec | | | |
| ″ | Tec | | | |
| ″ | Tie | | | |
| ″ | TK-Unique | | | |
| ″ | Trk | | | |
| ″ | VEGFR | | | |
| Other | AUR | | | |
| ″ | BUB | | | |
| ″ | BUD32 | | | |
| ″ | CAMKK | | | |
| ″ | CDC7 | | | |
| ″ | CK2 | | | |
| ″ | Haspin | | | |
| ″ | IKK | | | |
| ″ | IRE | | | |
| ″ | MOS | | | |
| ″ | NAK | | | |
| ″ | NEK | | | |
| ″ | NKF1 | | | |
| ″ | NKF2 | | | |
| ″ | NKF3 | | | |
| ″ | NKF4 | | | |
| ″ | NKF5 | | | |
| ″ | NRBP | | | |
| ″ | Other- | | | |
| | Unique | | | |
| ″ | PEK | | | |
| ″ | PLK | | Plk1 | Elia et al. (2003) |
| ″ | SCY1 | | | |
| ″ | Slob | | | |
| ″ | TBCK | | | |
| ″ | TLK | | | |
| ″ | TOPK | | | |
| ″ | TTK | | | |
| ″ | ULK | | | |
| ″ | VPS15 | | | |
| ″ | WEE | | | |
| ″ | Wnk | | | |
| Atypical | ABC1 | | | |
| ″ | Alpha | | | |
| ″ | Brd | | | |
| ″ | PDHK | | | |
| ″ | PIKK | | mTOR | Schalm & Blenis (2002) |
| ″ | RIO | | | |
| ″ | TIF1 | | | |

¹One or more references are provided for those families of kinases that are known to bind to one or more of their substrates via docking domains on native protein substrates. No attempt has been made to list references exhaustively.
²Only select sub-families are listed.
³The “reference” (Col. 5) describes a kinase substrate that contains a docking domain for a particular kinase (Col. 4).

Synthetic molecules are useful tools to evaluate protein kinases belonging to different enzyme families. An important issue with synthetic molecules that are used as substrates for intracellular enzymes is the fidelity with which such a peptide is acted upon by a specific enzyme. The primary sequence surrounding the site in the substrate where a reaction catalyzed by an enzyme takes place has been shown to be important in conferring substrate specificity for kinases. For example, a number of synthetic peptide substrates having 10-20 amino acids which correspond to the amino acid sequence surrounding the phosphoacceptor site can be acted upon efficiently and specifically by their cognate enzymes, particularly for phosphorylation by certain serine/threonine kinases (e.g., protein kinase A, protein kinase B, protein kinase C, casein kinase II, and others). However, the synthetic substrates for other enzymes do not display similar favorable characteristics. For example, different tyrosine kinases recognize similar substrate peptides and different mitogen-activated protein kinases share a similar substrate consensus sequence in the form of the Ser/Thr-Pro motif. For these enzymes simple peptides containing only residues surrounding the phosphorylation site do not serve as specific and/or efficient substrates (Songyang et al., 1995, Tanoue et al., 2002).

### SUMMARY

In the first aspect, the present invention is a method of introducing a fluorescent label into a cell that includes exposing a reporter to the cell. The reporter contains a peptide substrate for an enzyme, a docking domain for the enzyme that is attached to the peptide substrate, a membrane traversing moiety, and the label.

In the second aspect, the present invention a method of measuring the activity of a protein kinase in a cell that includes: introducing a membrane traversing peptide conjugate into the cell; lysing the cell to produce a lysate; subjecting the lysate to electrophoresis to separate a labeled reporter which reacted with the protein kinase from the labeled reporter which was unreacted; detecting the labeled reporter; and determining an amount of labeled reporter which reacted with the protein kinase and an amount of labeled reporter which was unreacted. The membrane traversing peptide conjugate contains a reporter; a transduction domain that is attached to the reporter, and a label that is attached to reporter. The reporter contains a peptide substrate for a protein kinase and a docking domain for the protein kinase that is attached to the peptide substrate.

In a third aspect, is a reporter that includes a peptide substrate for an enzyme and a docking domain for the enzyme that is attached to the peptide substrate. The enzyme is a protein kinase or phosphatase.

In a fourth aspect, the present invention is a reporter that includes a substrate for an enzyme and a docking domain for the enzyme that is attached to the substrate.

Definitions

Amino acid residues are referred to herein by their standard single-letter or three-letter notations or by their full names: A, Ala, alanine; C, Cys, cysteine; D, Asp, aspartic acid; E, Glu, glutamic acid; F, Phe, phenylalanine; G, Gly, glycine; H, His, histidine; I, Ile, isoleucine; K, Lys, lysine; L, Leu, leucine; M, Met, methionine; N, Asn, asparagine; P, Pro, proline; Q, Gln, glutamine; R, Arg, arginine; S, Ser, serine; T, Thr, threonine; V, Val, valine; W, Trp, tryptophan; Y, Tyr, tyrosine. The abbreviation “X” or “x” represents any amino acid and the abbreviation “Hyp” denotes hydroxyproline

The phrase “docking domain” refers to a polypeptide sequence that displays the following properties: (1) it contains at least 4 contiguous amino acid residues; (2) it must be able to bind to at least one enzyme with a dissociation contant (K_{d}) of less than 1 mM; (4) its presence in enzyme-substrate mixtures inhibits activity of the enzyme for native substrates with an inhibitory concentration-50 (IC₅₀) of less than 1 mM; and (5) preferably, its presence in a reporter increases by at least 2-fold the enzyme's ability utilize a reporter containing a synthetic peptide substrate for the enzyme relative to a synthetic peptide substrate alone.

The term “reporter” refers to a molecule that can serve as an enzyme substrate and which can be analyzed to determine the catalytic activity of an enzyme. As described herein, a reporter is a synthetic molecule containing a substrate for a particular enzyme and a docking domain for that enzyme. For example, a peptide substrate is an non-native, unnatural substrate for a kinase; i.e., the peptide substrate is not the full-length protein substrate. The peptide substrate can be a synthetic peptide that is produced using solid-phase chemistry methods, a recombinant peptide expressed from a cell, or a peptide generated by in vitro translation. A reporter may contain a covalently attached label.

The term “enzyme substrate” refers to a substrate for an enzyme-catalyzed reaction. Typical enzyme substrates include, but are not limited to, polypeptides, nucleic acids, polysaccharides, lipids, small organic molecules, macromolecules, biologically active agents, therapeutically active agents, agriculturally active agents, etc. Typical enzymes include proteins, and nucleic acid, such as catalytic DNA molecules, catalytic RNA molecules or ribozymes.

The term “label” refers to any moiety that is capable of detection, selection, or amplification, such as a radioactive element, a fluorescent moiety, a phosphorescent moiety, a luminescent moiety, a chemiluminescent moiety, metal coordination group (for example, a group that becomes fluorescent after metal or ion coordinates), an epitope for an antibody (which may be detected by reaction with a fluorescently labeled antibody), etc.

The term “peptide” as used herein refers to a compound made up of a single chain of D- or L-amino acids or a mixture of D- and L-amino acids joined by peptide bonds. Preferably, peptides contain at least two amino acid residues and are less than about 50 amino acids in length.

The term “protein” as used herein refers to a compound that is composed of linearly arranged amino acids linked by peptide bonds, but in contrast to peptides, has a well-defined conformation. Proteins, as opposed to peptides, preferably contain chains of 50 or more amino acids.

“Polypeptide” as used herein refers to a polymer of at least two amino acid residues and which contains one or more peptide bonds. “Polypeptide” encompasses peptides and proteins, regardless of whether the polypeptide has a well-defined conformation.

The term “peptide conjugate” refers to any peptide that is attached to another molecule, such as a label, another peptide, a polypeptide, an amino acid, a polysaccharide, a nucleic acid, etc.

The term “membrane traversing peptide conjugate” means that some membrane must be available for which the peptide conjugate is capable of traversing.

The term “membrane traversing moiety” refers to any molecule capable of mediating transmembrane transport of themselves and any cargo attached to their structure. A membrane traversing moiety means that some membrane must be available for which the moiety is capable of traversing. Typical examples of membrane traversing moieties include peptide transduction domains, myristoyl moiety, lipids, folate and the like.

The term “target peptide” refers to a peptide as a reactant for a chemical reaction occurring in a cell, such as an enzyme-catalyzed reaction, a nucleic acid binding reaction, a polysaccharide binding reaction, a polypeptide binding reaction, etc.

The phrases “peptide transduction domain” and “cell-penetrating peptide” refer to peptides capable of mediating transmembrane transport of themselves and any cargo attached to their structure, and are described in U.S. patent application Ser. No. 60/530,875 (Attorney Docket No. 60021010-0025), titled “A CELL-PERMEABLE ENZYME ACTIVATION REPORTER THAT CAN BE LOADED IN A HIGH THROUGHPUT AND GENTLE MANNER” to Allbritton et al. filed on Dec. 17, 2003

The term “transduction domain” has the same meaning as described herein for peptide transduction domain, or cell-penetrating peptide.

The term “cargo” refers to a molecule, such as a peptide, a target peptide, a polynucleotide, a ligand, a reporter, or an enzyme substrate that is initially attached to a transduction domain for delivery across a biological membrane.

The phrase “cargo-PTD conjugate” refers to a molecule containing a peptide transduction domain attached to a cargo molecule through at least one covalent bond.

The term “photolabile linkage” refers to a site of covalent attachment that is susceptible to cleavage following irradiation with light. An example of a photolabile linkage is one formed from an Fmoc-aminoethyl photolabile linker, such as 4-[4-(1-(Fmoc-amino)ethyl)-2-methoxy-5-nitrophenoxy]butanoic acid. Other examples of photolabile linkages are described by U.S. Pat. No. 5,917,016 titled “PHOTOLABILE COMPOUNDS AND METHODS FOR THEIR USE” to Christopher P. Holmes, which issued on Jun. 29, 1999.

### BRIEF DESCRIPTION OF THE DRAWINGS

FIG. 1A illustrates the results of ERK2-dependent phosphorylation of immobilized reporters that contain an intact docking domain and peptide substrate (Pn-ds and ds-Pn) and of immobilized reporters that contain a mutant docking domain (Pn-dsMUT and dsMUT-Pn) or a mutant peptide substrate (PnMUT-ds and ds-PnMUT);

FIG. 1B illustrates the average percent phosphorylation of these reporters relative to Pn-ds (N represents the number of experiments performed to calculate the average);

FIG. 2A shows a SDS-PAGE assay for the ability of ERK2 to convert MEK2 to phospho-MEK2 as a function of increasing concentrations of synthetic peptides containing either an intact docking domain (Ste7, MEK1 or MEK2) or a mutant docking domain (MEK2_{EEAA});

FIG. 2B is a graphical representation of percent MEK2 activity as a function of increasing synthetic peptide concentration;

FIG. 3 depicts the effect of ERK2 activity for phosphorylating Elk-1 by the presence of increasing concentrations of synthetic peptides that contain either an intact docking domain (Elk-1, MEK1, MEK2, or Ste7) or a mutant docking domain (Elk-1_{EEG}, MEK2_{EEAA}, or scram7).

FIG. 4 shows a Western Blotting assay using an anti-(phospho-ERK2) antibody to detect the ability of MKP-1 to convert phospho-ERK2 to ERK2 as a function of increasing concentrations of synthetic peptides containing either an intact docking domain (FIG. 4A: Ste7 and MEK2; FIG. 4B: Ste7, Elk-1, MKP-2, MEK-1 and MEK2) or a mutant docking domain (FIG. 4B: scram7, Elk-1_{EEG}, and MEK2_{EEAA}) at the indicated concentrations of added synthetic peptides;

FIG. 5A illustrates the results of a SDS-PAGE assay to detect ³⁵S-labeled MEK2 that precipitated with immobilized GST-ERK2 following incubation of the labeled MEK2 protein with GST-ERK2 in the presence of increasing concentrations of synthetic peptides containing either an intact docking domain (Ste7, MEK1, MEK2, or Elk-1) or a mutant docking domain (scram7, MEK2_{EEAA} (M2_{EEAA}), or Elk-1_{EEG} (Elk_{EEG}); and

FIG. 5B illustrates a graphical depiction of percent MEK2 precipitated as a function of the indicated concentrations of the synthetic peptides. The control protein, MEK2_{Δ4-16}, which lacks the MEK2 docking domain, did not bind to GST-ERK2 as revealed by the SDS-PAGE assay.

### DETAILED DESCRIPTION

The present invention makes use of the discovery that the specificity and efficiency of enzyme activity for artificial substrates can be dramatically improved by physically connecting a synthetic peptide substrate to a docking domain specific for a particular enzyme. The advantage of adding these domains to synthetic peptide molecules is that new reporters can be made that will function as more efficient and/or specific substrates for the enzymes whose activity it is desired to measure. This will increase the breadth of enzymes for which reporters can be designed and will increase the accuracy of these synthetic molecules as reporters of the activity of particular enzymes, especially for complex mixtures of enzymes, such as that found in cells.

The overall strategy is based on designing reporters composed of synthetic peptide substrates linked to docking domains for the enzyme of interest. The reporter is then introduced into cells in vivo or incubated with complex enzyme mixtures in vitro to permit reaction with the enzyme of interest. After a period of time in which the enzyme(s) is allowed to act upon the reporter, the reporter is analyzed to determine if the catalytic reaction has taken place. This analysis provides a measure of the activity of the enzyme(s) within the cell or in the complex enzyme mixture. The remainder of the specification illustrates various aspects of the invention by providing a description and examples of reporter molecules designed for detect protein kinases and protein phosphatases. Although the description is specific for kinases and phosphatases, it is applicable to any enzyme.

Reporter Design

Preferably, the reporter contains a peptide bond between the peptide substrate and the docking domain. Such reporter designs are preferred because the entire reporter may be synthesized as one continuous polypeptide with commercially available automated peptide synthesizers using well-established solid phase chemical methods, such as Fmoc-based chemistries. Optionally, the peptide substrate and the docking domain may be individually synthesized as separate molecules and ligated together following their purification. This alternative strategy is preferred in those cases where longer peptide substrates or longer docking domains are desired and whose combined length might be difficult to achieve efficiently in a single synthesis procedure using conventional solid phase methods. Optionally, longer peptides, particularly those encoding extensive docking domains (e.g., a docking domain with a length greater than 50 amino acids) can be expressed as recombinant peptides in vivo or translated in vitro and purified subsequently.

Optionally, the reporter contains the docking domain linked C-terminal to the peptide substrate. Optionally, the design of the reporter contains the docking domain linked N-terminal to the peptide substrate. Preferably, the arrangement of the docking domain to the peptide substrate mimics the arrangement of the enzyme's natural protein substrate that contains both the substrate and the docking domain. Even more preferably, the arrangement of the docking domain to the peptide substrate yields the greatest reporter activity for a particular enzyme of interest. One useful test of the optimal arrangement of the reporter is to ascertain whether enzyme utilization of a reporter is greater with one ordered arrangement of the docking domain and peptide substrate relative to their arrangement in reverse order.

Optionally, the peptide substrate may be attached to the docking domain by a linker. Where amino acid-based linkers are included in a reporter, preferred lengths of linkers range from 1-10 amino acids; more preferably, the linker length ranges from 3-8 amino acids; most preferably, the linker length ranges from 5-7 amino acids. The length of the linker can also be measured as the number of atoms between the docking domain and the synthetic peptide substrate; preferably, the length ranges from 3-30 atoms; more preferably, the length ranges from 9-24 atoms; still more preferably, the length ranges from 15-21 atoms. Preferred compositions of amino acid-based linkers are those that favor random coil configurations so as to permit the greatest flexibility of polypeptide conformation for the reporter. An example of a linker that adopts a random coil configuration is polyglycine. Linkers composed of other polymeric molecules are possible such as poly(8-amino-3,6-dioxo-octanoic acid) and poly(ethylene glycol).

Preferably, the reporter contains a label covalently attached to a specific site on its structure. The label may be any moiety that is capable of detection, selection, or amplification. Preferred labels include one of the following: a radioactive element (e.g., ³²P), a fluorescent moiety (e.g., fluorescein), a phosphorescent moiety, a luminescent moiety, a chemiluminescent moiety, metal coordination group (e.g., a group that becomes fluorescent after metal or ion coordinates), an epitope for an antibody (which may be detected by reaction with a fluorescently-labeled antibody), etc. Optionally, the label may be covalently attached to either the peptide substrate portion or the docking domain portion of the reporter. Optionally, the label may be covalently attached to either the N-terminus or the C-terminus of the reporter. Optionally, the label may be covalently attached to a side-chain group of an amino acid within the reporter. The only requirement that the label attachment site must satisfy is that the presence of the label on the reporter does not compromise reporter activity. A useful test of whether inclusion of a label on a reporter compromises reporter activity is to ascertain whether enzyme utilization of a reporter is inhibited by the presence of the label as compared with a reporter lacking the label.

The synthetic peptide substrate portion of the reporter contains the site of covalent modification by the enzyme of interest and optionally one or two additional flanking amino acid sequences that surround the site of covalent modification. Preferably, the flanking amino acid sequences each contain from 1-8 amino acids. More preferably, the flanking amino acid sequences each contain 5-6 amino acids. Most preferably, the flanking amino acid sequences each contain 5-6 amino acids that correspond to the natural amino acid sequence of the protein substrate for the particular enzyme of interest.

The reporter can be modified to include additional moieties, such as a phosphate moiety, a myristoyl moiety, a lipid moiety, a carbohydrate moiety, a sugar moiety (e.g., ribose, fructose, glucose, etc.), a sulfate moiety, a biotin moiety, a coordination group moiety (e.g., heme, porphyrin, EDTA, etc.), a ubiquitin moiety, a nucleobase-containing moiety (e.g., NAD, NADP, FAD, cAMP, ATP, GTP, CTP, UTP, TTP, etc.) and the like. Such moieties may be attached to the docking domain, the peptide substrate or both. Optionally, such moieties may serves as synthetic substrates in those reporters designed to detect enzymes that act upon the moieties as substrates.

Intracellular Enzyme Activity Measurements using Reporters

In cases where the enzyme activity measurements are sought in intact cells, the reporter must cross the plasma membrane or otherwise enter the cell. Preferably, a reporter can contain a membrane traversing moiety. Examples of membrane traversing moieties include peptide transducing domains, myristoyl moiety, folate and the like. Each of these membrane traversing moieties will be described below.

A novel class of polypeptides, termed peptide transducing domains (PTDs), has been discovered to facilitate transmembrane transport of attached exogenous cargo into the cell. Examples of PTDs include Arg₉, the D-isomeric form of Arg₉, Penetratin, basic fragments from HIV-1 Tat protein (residues positions 48-60), signal-sequence-based peptides, Transportan, and the like (Lindgren et al., 2000).

In the present invention, a reporter may be attached to a PTD to form a reporter-PTD conjugate so that the reporter can be transported across the otherwise impermeable cellular membrane. Cells are bathed in solutions of the reporter-PTD conjugate, permitting transport across the cellular membrane. The remaining extracellular peptides are washed away, allowing subsequent investigation of intracellular enzymes.

The use of reporter-PTD conjugates provides for high throughput loading of reporters into cells without physically damaging the cells. The use of reporter-PTD conjugates may save considerable money in that no additional equipment (such as a microinjector, pulsed laser, or electric field generator) is required and smaller amounts of loading material are typically used. Additionally, the use of reporter-PTD conjugates does not require specialized skills and can be done by simply exchanging solutions. Further, reporter-PTD conjugates can potentially be used to exact greater control over the availability of the reporter within the cell. This includes using a photo-cleavable caging mechanism to cleave the PTD from the reporter. Moreover, PTD-mediated loading results in less cellular damage.

The PTD can be attached to the reporter by a cleavable or non-cleavable linkage. A cleavable linkage is particularly useful when a covalently attached PTD negatively impacts the interaction of the reporter with the enzyme of interest, or when the properties of the PTD make subsequent analysis of the reporter-PTD conjugate difficult. Examples of cleavable linkages include disulfide bonds that can be broken by intracellular glutathione, and photoreactive linkers such as 4-[4-(1-ethyl)-2-methoxy-5-nitrophenoxy] butanoic acid that can be cleaved by the appropriate frequency of light.

The preparation of PTD conjugates and methods for their use have been described in U.S. patent application Ser. No. 60/530,875 (Attorney Docket No. 60021010-0025), titled “A CELL-PERMEABLE ENZYME ACTIVATION REPORTER THAT CAN BE LOADED IN A HIGH THROUGHPUT AND GENTLE MANNER” TO Allbritton et al., filed on Dec. 17, 2003.

A prophetic example describes the use of reporter-PTD conjugates in connection with laser-assisted cell lysis and capillary electrophoresis to monitor intracellular enzyme activity of non-native reporters. This approach is described in U.S. Pat. No. 6,335,201, titled “METHOD AND APPARATUS FOR DETECTING ENZYMATIC ACTIVITY USING MOLECULES THAT CHANGE ELECTROPHORETIC MOBILITY” to Allbritton et al., which issued on Jan. 1, 2002.

A number of approaches can be taken to transfer the reporter into cells in addition to conjugation of a protein transduction domain to the reporter. These approaches include the addition of a lipid moiety such as a myristoyl group to the reporter, or the addition of folate (Leamon & Low, 2001). The association of the reporter with a noncovalent carrier such as liposomes or Pep-1 can also be employed (Bonetta, 2002). Physical methods can also be used to transfer the reporter into the cell including microinjection, electroporation, microprojectile bombardment, optoporation, osmotic shock, etc. (Stephens & Pepperkok, 2001; Soughayer et al., 2000; Okada & Rechsteiner, 1982).

Identification of Activities for Protein Kinase Families using Designer Reporters

Docking domains for an enzyme belonging to a protein kinase family may be used in a reporter to detect other members of the family. Docking domains have already been well characterized for six of the nine known groups of protein kinases, including GMGC, STE, AGC, TK, Other, and Atypical (see Table 1). Reports describing these docking domains are hereby incorporated by reference (Adams et al., 1996; Zheleva et al., 2002; Dajani et al., 2001; Bardwell et al., 2001; Yang et al., 1998; Kallunki et al., 1996; Ho et al., 2003; Enslen et al., 2000; Yang et al., 1999; Tanoue et al., 2000; Rubinfeld et al., 1999; Biondi, 2002; Clark et al., (2001); Duyster et al., 1995; Guappone & Flynn, 1997; Mayer et al., 1991; Mayer et al., 1995; Koch et al., 1989; Elia et al., 2003; Schalm & Blenis, 2002). In this manner, entire groups of kinases may be surveyed in both their natural intracellular environments and complex mixtures using reporters that have enhanced efficiency and specificity for their cognate enzymes.

One prophetic example describes a reporter for the Abl and Bcr/Abl kinases that uses a similar approach to that described above for reporters directed to MAPK activities (see Example 6). The Abl kinase portion of the Bcr-Abl fusion protein contains both SH3 and SH2 domains. These domains of approximately 60 and 100 amino acids in length mediate protein-protein interactions in many signaling pathways (Smithgall 1995). SH3 domains bind to relatively short consensus sequences in target proteins rich in proline and hydrophobic amino acids, while SH2 domains recognize and bind to phosphotyrosine residues in their target (Birge & Hanafusa, 1993; Ren et al., 1993). The binding specificity of an SH2 domain appears to be determined primarily by the three amino acids C-terminal to the tyrosine, and short peptides (10-12 residues) containing a —Y(PO₃)-E-N—P— sequence have been shown to bind to the SH2 domain of Abl with affinities in the 1-50 nM range and with high specificity (Songyang et al., 1993). Thus, these docking domains differ markedly from those present in other protein kinase substrates. A fusion peptide composed of the SH2 binding domain (GDGY_{(PO3)}ENPSP) (Songyang et al., 1993) with a peptide substrate (EAIYAAPFA) (Songyang et al., 1995) is expected to be a reporter selective for measuring the activities of the Abl and Bcr/Abl kinases.

Diagnostic Applications of Reporter and Reporter-PTD Conjugates

The finding that an adjacent MAPK docking domain could stimulate the phosphorylation of a near-optimal peptide substrate was unexpected. In particular, reporter designs are feasible where the natural length of amino acid sequence that separates docking domains from substrate phosphorylation sites in the native protein kinase substrates can be substantially reduced, if not altogether eliminated. Shortened reporter designs are preferred from the standpoints of cost of production, solubility, stability and suitability for inclusion in larger reporter designs, such as reporter-PTD conjugate designs.

Reporters are useful as reagents. For example, efficient and specific MAP kinase substrate peptides would be advantageous in a recently developed methodology for monitoring the activity of multiple kinases in single cells (Meredith et al., 2000). These types of reporters may be used in applications for characterizing the molecular profile of MAPK activities within particular types of diseases. For example, different types of lymphomas may be readily cataloged based upon their expression profile of MAPK activities.

More generally, however, reporters possessing docking domains specific for different protein kinase families are powerful diagnostic tools to characterize normal and neoplastic cells. Such tools would afford the clinician the ability to diagnose different diseases, to detect stages of a particular form of disease, and to monitor the clinical efficacy of therapeutic compounds designed to treat such conditions. For example, the clinician can use enzyme activity assays that employ different reporters. One collection of reporters may be designed that is specific for a group of protein kinases (e.g., GMGC) or phosphatases (e.g., Shp). Another collectoin of reporter may contain cell-specific PTDs that permits selective targeting of specific cell types or stages of cellular differentiation. The clinician can analyze a small blood or tissue sample from a patient using the chosen reporter-PTD conjugates in the following manner: (1) contacting the individual conjugates to the patient sample, (2) permitting the reporter-PTD conjugates to enter cells and undergo reaction with the enzyme of interest; and (3) determining the activity of the enzyme using a technique such as capillary electrophoresis. These techniques and others are described in U.S. patent application Ser. No. 09/859,650, titled METHOD AND APPARATUS FOR DETECTING CANCEROUS CELLS USING MOLECULES THAT CHANGE ELECTROPHORETIC MOBILITY to Allbritton & Sims, filed May 17, 2001, published as U.S. Patent Application Publication No. 20020037542 on Mar. 28, 2002, which is hereby incorporated by reference.

Use of a Reporter to Enhance the Efficiency and Specificity of Protein Kinase for a Synthetic Substrate

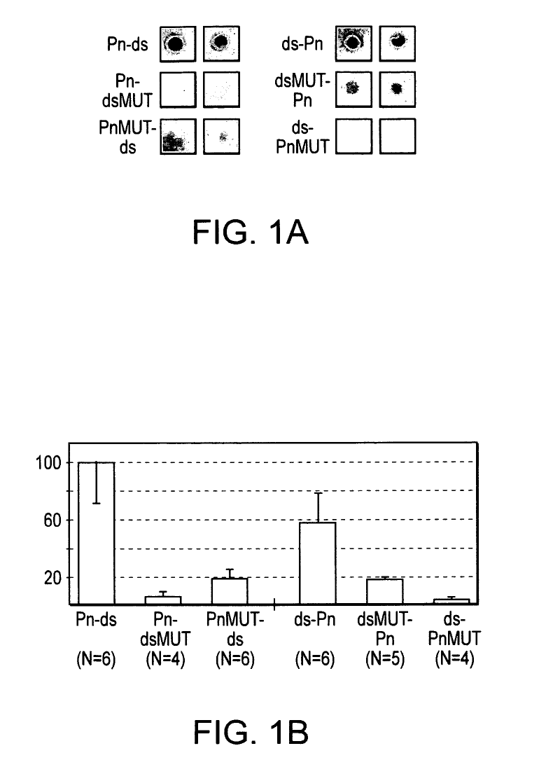

For the design of a reporter for detecting ERK activity, peptide substrate (P/G-GPLSPGGG) was chosen from a consensus sequence for ERK1 (Songyang et al., 1996). A docking domain (KKKPTPIQLNPAP) was chosen from the docking domain of MEK1 (Bardwell & Thorner, 1996; Bardwell et al., 2001). The target phosphorylation site chosen contains optimal residues for ERK-mediated phosphorylation at the +1, +2, −1, −2 and −3 positions, and also contains a preferred residue at the +3 position (Songyang et al., 1996). The resultant reporter molecule was synthesized using solid-phase chemistry procedures (see Example 8, Table 2 (“Pn-ds” and “ds-Pn”)). The requirement of a MAPK docking domain for efficient ERK2-mediated phosphorylation was demonstrated by the use of a peptide array phosphorylation assay (FIG. 1). In this assay, different reporters were synthesized and subsequently anchored to a cellulose membrane. The filter was incubated with a solution containing active ERK2 and [γ-³²P]ATP, and phosphate incorporation into the individual peptide spots was quantified. Only peptides containing an intact MAPK docking domain, regardless of the presence of a high efficiency phosphoacceptor site, were phosphorylated effectively by active ERK2.

Docking Domain Peptides Inhibit Activities of Enzymes Belonging to the Same Protein Kinase Family

Several studies have demonstrated the presence of MAPK docking domains in the N-terminal regions of MEKs that are important for both high-affinity binding to, and phosphorylation of, target MAPKs (Bardwell et al., 1996; Fukuda et al., 1997; Xia et al., 1998; Kieran et al., 1999; Enslen et al., 2000; Tanoue et al., 2000; Bardwell et al., 2001). The core of these docking domains is a sequence with the consensus (R/K)₂-(X)₂₋₆-L/I-X-L/I (Bardwell & Thomer, 1996; Holland & Cooper, 1999; Sharrocks et al., 2000); mutation of either the basic or hydrophobic residues in this motif results in impaired MAPK binding and activation (Enslen et al., 2000; Tanoue et al., 2000; Bardwell et al., 2001; Xu et al., 2001). Furthermore, synthetic peptides corresponding to this MAPK docking domain from MEKs can inhibit MEK1, MKK3 or MKK6 binding to, and activation of, their cognate MAPKs (Enslen et al., 2000; Tanoue et al., 2000; Bardwell et al., 2001; Xu et al., 2001) presumably because the peptides are able to compete with the MEKs for binding to MAPKs.

MAPK docking domains in MEK1 and MEK2 proteins are both necessary and sufficient for the formation of stable protein complexes with ERK1 and ERK2 (Bardwell et al., 2001). Upon activation, MEK1 and MEK2 activate ERK1 and ERK2 by dual phosphorylation of the threonine and tyrosine residues within a Thr-Glu-Tyr motif. In order to examine the importance of the MAPK docking domain in MEK2 for its ability to phosphorylate ERK2, synthetic peptides corresponding to the MAPK docking domains of MEKs were tested for their ability to inhibit MEK2 phosphorylation of ERK2. The peptides used were 17-21 residues in length (see Example 8, Table 2).

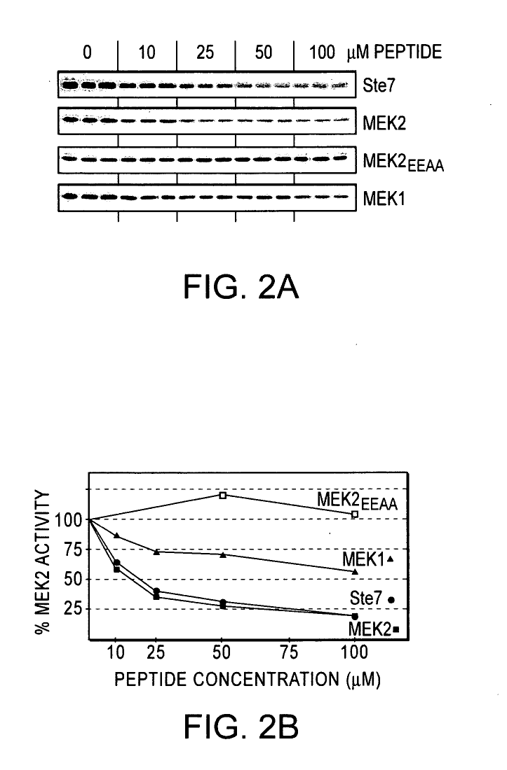

Kinase assays contained active MEK2 and inactive ERK2 as substrate. When short synthethic peptides corresponding to the MAPK docking domains of MEK1, MEK2 or Ste7 were titrated into the MEK2 kinase assays, a dose-dependent inhibition of ERK2 phosphorylation was observed (FIG. 2). Thus the MAPK docking domain of MEK2 is not only important to binding to ERK2 (Bardwell et al., 2001), but also, MEK2-ERK2 interaction via this domain facilitates ERK2 phosphorylation. In contrast, a MEK2 mutant peptide bearing mutations in the basic amino acid residues and hydrophobic amino acid residues did not inhibit the ability of MEK2 to phosphorylate ERK2 (see FIG. 2). This confirms the requirement for these conserved residues for efficient ERK2 interaction.

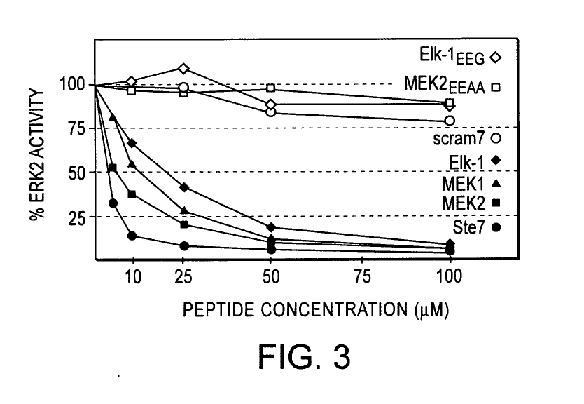

MEK1 and MEK2 are closely related proteins, both of which target ERK1 and ERK2 in cells. Intriguingly, the docking domains of MEK1 and MEK2 show less sequence conservation then the rest of these proteins. The IC₅₀ of the MEK1 and MEK2 docking domain peptides used in these experiments were approximately 100 and 20 μM, respectively (see FIG. 2). This indicates that the MEK2-ERK docking interaction is stronger than the MEK1-ERK interaction, consistent with previous work (Bardwell et al., 2001). Ste7 is a MEK that phosphorylates Kss1 and Fus3 in the yeast S. cerevisiae, and was the first MEK found to contain a MAPK docking domain (Bardwell & Thomer, 1996; Bardwell et al., 1996). Interestingly the Ste7 docking domain peptide is a very effective inhibitor of ERK2 phosphorylation by MEK2 with an IC₅₀ of approximately 20 μM (see FIG. 2). These docking domain peptides were also shown to inhibit ERK2-dependent phosphorylation of Elk-1, whereas mutant docking domain peptides were ineffective inhibitors of ERK2 activity (see Example 3, FIG. 3).

MKPs, a class of dual-specificity phosphatases that act on MAPKs, remove phosphate groups from both the threonine and tyrosine residues in the Thr-X-Tyr motif (Keyse 2000). Some MKPs such as MKP-3, MKP-4, MKP-5 and MKP-7 are located predominately in the cytoplasm, while MKP-1 and MKP-2 are inducible, nuclear enzymes (Keyse 2000). Several of the MKPs have been shown to contain a MEK-like docking domain important for MAPK-binding (Tanoue et al., 2000; Muda et al., 1998; Chen et al., 2001; Farooq et al., 2001; Slack et al., 2001; Zhou et al., 2001; Tanoue et al., 2002).

The MAPK docking domain in either MKP-1 or MKP-2 has been shown to be critical for optimal ERK2 binding and mutation of this domain also severely compromises ERK2-dependent activation of MKP-1 and MKP-2 catalytic activity (Chen et al., 2001; Slack et al., 2001). Since the MAPK-docking domains in these MKPs approximate the same consensus as those in the MEKs, it is possible that these ERK-interacting MKPs utilize this docking domain to compete with MEKs for binding to the same domain on their MAPK targets. In order to test this hypothesis, a synthetic peptide corresponding to the MAPK-docking domain region of MKP-2 was tested for its ability to inhibit MEK2 phosphorylation of ERK2. The MKP-2 peptide inhibited MEK2 phosphorylation of ERK2 in a dose-dependent manner (IC₅₀-50 μM), presumably by competing for the same binding domain on ERK2. On the other hand, a peptide in which the three conserved arginine residues and the two conserved hydrophobic residues in the MKP-2 MAPK docking domain were mutated, did not inhibit MEK2 phosphorylation of ERKs (less than 20% inhibition at 100 μM peptide).

If the MAPK docking domains on MKP-2 and Elk-1 compete for binding to ERK2, then the MKP-2 peptide should inhibit ERK2 phosphorylation of Elk-1. Indeed, when the MKP-2 peptide was titrated into an ERK2 kinase assay utilizing GST-Elk-1 as substrate, the MKP-2 peptide inhibited ERK2 phosphorylation of Elk-1 in a dose-dependent manner (IC₅₀-20 μM) while the corresponding mutant forms of docking domain peptides for MKP-2 had no effect on Elk-1 phosphorylation.

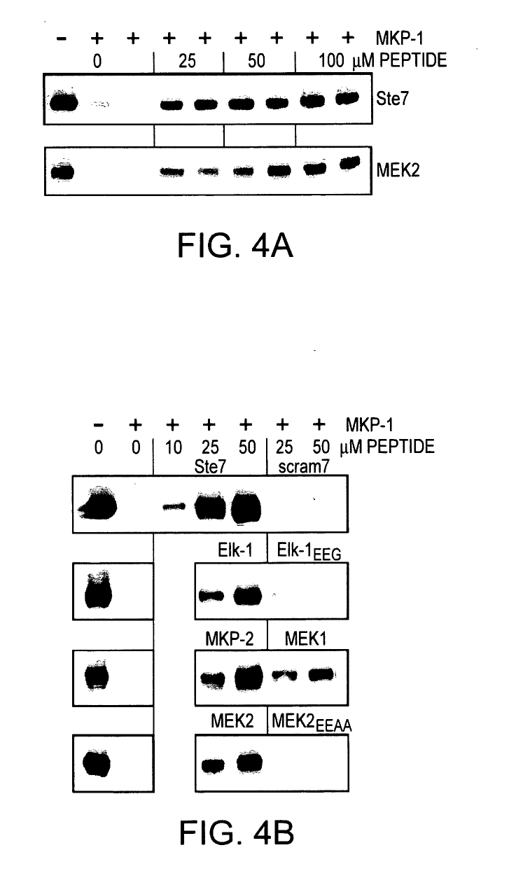

If the MAPK docking domains on MEKs, MKPs and Elk-1 compete for binding to ERK2, then the MEK and Elk-1 peptides should inhibit MKP-mediated dephosphorylation of ERK2. To explore this possibility, a series of MKP-1 phosphatase assays were performed utilizing activated ERKS as substrate, in the presence or absence of peptide. MKP-1, like MKP-2, is an inducible, nuclear dual-specificity phosphatase that can utilize ERK2 as substrate (Keyse, 2000; Slack et al., 2001); MKP-1 and MKP-2 share a high level of sequence similarity, including their MEK-like docking domain regions. Phosphatase assay products were resolved on SDS-PAGE gels, transferred to membranes, and an antibody specific for phospho-ERK2 was used to detect remaining active ERK2. Under the reaction conditions used, MKP-1 dephosphorylated ERK2 completely when peptide was omitted (see FIG. 4). In contrast, a dose-dependent inhibition of MKP-1 mediated dephosphorylation of phospho-ERK2 was observed in the presence of the Ste7, MEK2, MEK1, Elk-1 and MKP-2 peptides. The mutant forms of the Elk-1 and MEK2 docking domain peptides did not inhibit MKP-1 dephosphorylation of ERK2 (see FIG. 4).

Docking Domain Peptides Inhibit Binding of a Substrate to its Enzyme

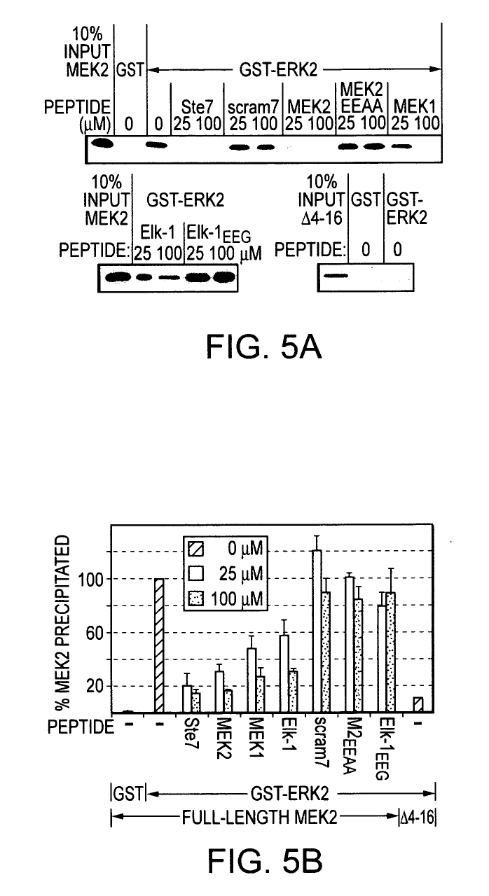

In order to demonstrate directly that the peptides were inhibiting stable protein complex formation between MEK2 and ERK2, a series of binding assays were performed. The ability of radiolabeled, in vitro-translated, full-length MEK2 protein to co-sediment with GST-ERK2 bound to glutathione-sepharose beads were tested in the presence or absence of the various MAPK-docking domain peptides. MEK2 protein co-sedimented with GST-ERK2 in the absence of docking domain peptides (see FIG. 5; Bardwell et al., 2001). This binding was specific because precipitation of MEK2 protein did not occur when GST was used instead of the GST-ERK2 fusion protein, regardless of whether the peptides were included in the reactions (see FIG. 5). When the Ste7, MEK2, MEK1, or Elk-1 docking domain peptides were individually added to the reactions, each docking domain peptide inhibited the amount of MEK2 protein that co-sedimented with GST-ERK2 (see FIG. 5). Thus, the MEK1, MEK2, Ste7 and (to a lesser extent) Elk-1 docking domain peptides are all able to compete with MEK2 protein for binding to ERK2.

The Ste7 and MEK2 docking domain peptides were the most potent inhibitors of MEK2 binding to GST-ERK2 (see FIG. 5). This correlates well with the finding that these peptides were also the strongest inhibitors of MEK2 phosphorylation of ERK2 (see FIG. 2). In fact, at 25 μM, the Ste7 peptide inhibited co-sedimentaiton of MEK2 with ERK2 to a similar degree as did complete removal of the docking domain from MEK2 (i.e., MEK2_{Δ4-6} protein, which lacks residues 4-16; see Example 8, Table 2). In contrast, the mutant forms of these peptides did not inhibit co-sedimentation of MEK2 protein with GST-ERK2. The correlation between the ability the peptides to block binding and to inhibit enzymatic activity strongly suggests that the enzymatic inhibition is a consequence of the blocking of docking.

### EXAMPLES

### Example 1

### A Reporter Containing a MAPK Docking Domain Potentiates ERK2 Kinase Activity

The purpose of this example is to determine whether a reporter containing both MAPK docking domain and a synthetic MAPK peptide substrate displays greater phosphorylation than reporters containing only the synthetic MAPK peptide substrate. A peptide array phosphorylation assay was used for this experiment. In this assay, a series of reporters in the form of short synthetic peptides were synthesized and subsequently anchored to a cellulose membrane. Each spot represents nanomolar quantities of a different reporter. Reporters were designed containing the MAPK docking domain of MEK1 immediately adjacent to a preferred target phosphorylation site (PLSP) for ERK2. The MAPK docking domain of MEK1 was chosen as the best compromise with regard to considerations of length, affinity and specificity. The target phosphorylation site chosen contains optimal residues for ERK-mediated phosphorylation at the +1, +2, −1, −2 and −3 positions, and also contains a preferred residue at the +3 position (Songyang et al., 1996). The reporters were synthesized in two different configurations with respect to the relative positioning of the phosphoacceptor site and the MAPK docking domain. Control reporters contained either a mutant, unphosphorylatable target site (PLAP) or a mutant form of the docking domain in which the key basic and hydrophobic residues had been mutated. The filter was incubated with a solution containing active ERK2 and [Y-³²P]ATP, and phosphate incorporation into the individual peptide spots was quantified.

A high level of peptide phosphorylation was seen for the peptides that contained the phosphoacceptor site together with the intact MAPK docking domain sequence, regardless of the orientation of the docking domain relative to the phosphoacceptor target site (see FIG. 1). Strikingly, considerably less peptide phosphorylation was observed with the peptides containing the mutant MAPK docking domain, despite that fact that these peptides still contained a near-optimal phosphoacceptor site sequence.

### Example 2

### Inhibition of MEK2-Dependent Phosphorylation of ERK2 by MEK Peptides

The following experiment was done to investigate whether docking domain peptides could inhibit MEK2 phosphorylation of ERK2. Purified, full-length, catalytically inactive ERK2 (1 μM) was incubated with 0.1 units of purified active MEK2 and [γ-³²P]ATP for 20 min, in the absence or presence of the specified concentrations of the docking domain peptides (MEK1, MEK2 or Ste7) or a mutant form of a docking domain peptide (MEK2_{EEAA}). An autoradiogram of a representative experiment is shown (see FIG. 2A). The results were plotted as percent phoshphorylation relative to that observed in the absence of any added peptide (see FIG. 2B). ERK2 phosphorylation was analyzed by SDS-PAGE and quantified. Data are the average of two to four experiments, with triplicate data points in each experiment.

### Example 3

### Inhibition of ERK2-Dependent Phosphorylation of Elk-1 by MEK and Elk-1 Docking Domain Peptides

The following experiment was done to ascertain whether MAPK docking domain peptides could inhibit ERK2 phosphorylation of Elk-1. Purified GST-Elk-1 (1 μM) was incubated with [γ-³²P]ATP and 10 units of purified, active ERK2 for 20 min, in the absence or presence of the specified concentrations of the indicated peptides. Elk-1 phosphorylation was analyzed by SDS-PAGE and quantified. Data are the average of three to eleven experiments, with triplicate data points in each experiment. The results of the phosphorylation assay were plotted as percent phosphorylation relative to that observed in the absence of any added peptide (see FIG. 3).

### Example 4

### Inhibition of MKP-1-Dependent Dephosphorylation of ERK2 by MEK, Elk-1 and MKP-2 Docking Domain Peptides

The following experiment was performed to assess whether MAPK-docking domain peptides would inhibit dephosphorylation of ERK2 by MKP-1. Purified, full-length, activated ERK2 (0.5 pmol) was incubated with 0.5 units of purified active MKP-1 for 20 min, in the absence or presence of the specified concentrations of the indicated peptides. ERK2 dephosphoryation was quantified by SDS-PAGE followed by western blotting and immonostaining with anti-phospho-ERK2 (Thr202/Tyr204). The lanes corresponding to ERK2 with no MKP-1 or no added peptide contain 50% of the relative amounts loaded in all other lanes (see FIG. 4A).

### Example 5

### Inhibition of MEK2 Binding to GST-ERK2 by MEK and Elk-1 Peptides

The purpose of the following experiment is to assess whether MAPK docking domain peptides would inhibit MEK2-binding to GST-ERK2. Radiolabeled (³⁵S) MEK2 and MEK2_{Δ4-16} proteins (˜1 pmol) were prepared by in vitro translation, partially purified by ammonium sulfate precipitation, and then incubated with 10 μg of purified GST or GST-ERK2 pre-bound to glutathione-Sepharose beads, in the absence or presence of the specified concentrations of docking domain peptides (Ste7, MEK1, MEK2, Elk-1) or mutant forms of docking domain peptides (scram7, MEK2_{EEAA}, Elk_{EEG}). Bead-bound protein complexes were isolated by sedimentation and resolved on 12% SDS-polyacrylamide gels. An autoradiogram of a representative experiment is illustrated (see FIG. 5A). Gels were analyzed by staining with Coomassie Blue for visualization of the bound GST fusion protein in order to verify equal amounts in each reaction and by autoradiography for visualization of the bound radiolabeled MEK2 or MEK2_{Δ4-16} to GST or GST-ERK2 in the absence of peptide (hatched bars), or in the presence of 25 μM (light gray bars) or 100 μM peptide (dark gray bars) (see FIG. 5B). Data are the average of three to five experiments; standard error bars are shown.

### Example 6

### A Reporter Containing an SH2 Docking Domain Potentiates Abl Kinase Activity (Prophetic Example)

The purpose of this example is to determine whether a reporter containing both an SH2 docking domain and an Abl kinase peptide substrate displays greater phosphorylation than reporters containing only an Abl kinase peptide substrate. A peptide array phosphorylation assay will be used for this experiment. In this assay, a series of reporters in the form of short synthetic peptides will be synthesized and subsequently anchored to a cellulose membrane. Each spot will represent nanomolar quantities of a different reporter. Reporters will be designed containing an SH2 docking domain (e.g., GDGY_{(PO3)}ENPSP; Songyang et al., 1993) immediately adjacent to a preferred target phosphorylation site for the Abl kinase (e.g., EAIYAAPFA; Songyang et al., 1995). The reporters will be synthesized in two different configurations with respect to the relative positioning of the phosphoacceptor site and the SH2 docking domain. An example of one such reporter is Pn(Abl)-ds(SH2) (see Example 8, Table 2). Control reporters will contain either a mutant, unphosphorylatable target site (e.g., Y→A mutation), an unphosphorylated Tyr in an SH docking domain, or a mutant form of the docking domain in which the key residues will be mutated (e.g., ENP→AAA mutation). The filter will be incubated with a solution containing [γ-³²P]ATP and active Abl kinase, and phosphate incorporation into the individual peptide spots will be quantified. A high level of peptide phosphorylation is expected for the peptides that contained the phosphoacceptor site together with the intact SH2 docking domain sequence.

### Example 7

### Assay of the Activity of an Intracellular Enzyme and Effects of Compounds Thereof (Prophetic Example)

A membrane traversing peptide is prepared which includes a label, such as a fluorescent molecule, attached to a reporter, such as a synthetic peptide substrate for a kinase and a docking domain for a particular kinase, which is in turn attached to a peptide transduction domain, for example by a photolabile linkage. A cell, such as a human cell, in then exposed to the membrane traversing peptide, causing the cell to take it up. Next, the cell is exposed to either a control compound or a compound, such as a drug candidate based upon a docking domain peptide. Optionally, the drug candidate may be a cargo-PTD, where the cargo includes the docking domain peptide attached to the PTD by a disulfide linker. The cell is then exposed to light to cleave the photolabile linkage; this activates the reporter for reaction with the kinase. Then the activity of the kinase is examined, for example by laser lysis of the cell and subsequent electrophoresis, to determine the ratio of unreacted target peptide to reacted target peptide. If the activity of the kinase is changed when the cell is exposed to the compound, this may be attributed to the effects of the compound. In this prophetic example, the cell could have been exposed to the compound before introducing the membrane traversing peptide.

### Example 8

### Experimental procedures for Examples 1-7

Kinase Assays

Kinase reactions (20 μl) for MEK2 phosphorylation of ERK2 contained kinase assay buffer (50 mM Tris-HCl (pH 7.5), 10 mM MgCl₂, 1 mM EGTA, 2 mM dithiothreitol (DTT)), 1 μM inactive mouse ERK2 (K52R mutation; New England Biolabs), 0.1 units active human MEK2 (Upstate Biotechnology), 50 μM ATP, 1 μCi [γ-³²P]ATP, and the indicated concentration of peptide. The K52R mutation in ERK2 lies within the ATP-binding pocket rendering it catalytically inactive. Thus, phosphorylation of ERK2 by MEK2 did not result in activated ERK2 that could autophosphorylate or back phophorylate MEK2 in the assays. ERK2 and peptide were pre-incubated in buffer for 10 min at 37° C., then returned to ice prior to the addition of ATP and MEK2. Reactions were for 20 min at 30° C. ERK2 phosphorylation was quantified by SDS-PAGE followed by analysis of relative incorporation using a Phosphorlmager™ (Molecular Dynamics, Inc.).

Kinase reactions (20 μl) for ERK2 phosphorylation of Elk-1 contained kinase assay buffer (see above), 1 μM GST-Elk-1 (a fusion protein consisting of residues 307-428 of human Elk-1 fused to GST; New England Biolabs), 10 units active mouse ERK2 (New England Biolabs), 50 μM ATP, 1 μCi [γ-³²P]ATP, and the indicated concentration of peptide. Reactions were for 20 min. at 30° C. Reactions were analyzed and quantified as above.

Protein Binding Assays

The constructs and methods used in the GST co-sedimentation (“pull-down”) experiments shown in FIG. 5 have been described elsewhere (Bardwell et al., 2001). The indicated concentrations of peptides were added to the GST-glutahione sepharose or GST-ERK2-gluththione-sepharose beads prior to the addition of the radiolabeled protein.

Dephosphorylation Assays

Phosphatase reactions (20 μl) for MKP-1 dephosphorylation of ERK2 contained 50 mM Tris-HCl (pH 7.5), 50 mM NaCl, 0.5 pmol active mouse ERK2, 0.5 units active human MKP-1 (Upstate Biotechnology) and the indicated concentration of peptide. Reactions were for 30 min. at 30° C. Reactions were analyzed by SDS-PAGE followed by western transfer and immunostaining with anti-phospho-ERK2 (Thr202/Tyro204) antibody (New England Biolabs).

Cellular Activity by Capillary Electrophoresis (CACE)

CACE has been previously described (Sims et al., 1998; Meredith et al., 2000; Li et al., 2001). Briefly, cells were placed on an inverted fluorescence microscope stage (Diaphot 300, Nikon, Japan) and maintained at 37° C. with an objective heater (Bioptechs; Butler, Pa.) prior to lysis unless stated otherwise. The cells were continually washed with ECB maintained at 37° C. (unless otherwise indicated) with a flow rate of 1 ml/min and a total chamber volume of 0.5 ml. The inlet of a capillary (30 μm I.D., 360 μm O.D., 75 cm long) was positioned 10 μm above the cell. The inverted microscope was coupled to a pulsed Nd:YAG laser and a CE system (Sims et al., 1998). The CACE-based assay was performed as previously described (Meredith et al., 2000; Li et al., 2001). Cells were always maintained in ECB when analyzed by the CACE.

Capillary Electrophoresis (CE).

CE was performed as described (Sims et al., 1998), with the following modifications. The inner walls of the capillaries (30 μm I.D., 360 μm O.D.) were coated with poly(acrylate) (Wang et al., 2003). The outlet of the capillary was held at a negative potential of 18-21 kV, and the inlet reservoir was held at ground potential. Under these conditions, the current through the capillary was typically ˜36 μA. Solutions of standards were loaded into the capillary by gravitational fluid flow. The loaded volume was calculated from Poiseulle's equation and from contributions by spontaneous fluid displacement and diffusion. To estimate the number of moles of peptide in an unknown solution or within a cell, the peak area from the electrophoretic trace of the solution or the cell was compared to that of a standard of known concentration separated by electrophoresis on the same day. A 1 pL cell volume and 100% peptide recovery was assumed in calculating intracellular peptide concentration (Sims et al., 1998; Meredith et al. 2000). Analytes were detected by laser-induced fluorescence (Sims et al., 1998).

Peptides and Peptide Array Assay

Soluble peptides used in this study were synthesized by United Biochemical Research, Inc. (Seattle, Wash., U.S.A.) and are presented in Table 2. Custom synthesis of the peptide arrays used in this study was performed by the ResGen division of Invitrogen Corp. (Carlsbad, Calif., U.S.A.). This technology has recently been reviewed (Reineke et al., 2001). Typically, a ˜13 cm² membrane containing 16 peptide spots was used in an experiment. The membrane was first blocked by incubation with 0.4 mL kinase assay buffer (see above) containing 1 mg/ml BSA and 50 μM ATP. The blocking solution was layered directly onto each peptide spot, ˜20/μl/spot. After 15 min at 30° C., this solution was removed completely by aspiration, and the membrane was then incubated with 0.4 μL of a mixture containing 0.5 μC/μl [γ-³²P]ATP, for 30 min at 30° C. The membrane was then washed 4 times 5 min in phosphate-buffered saline containing 5 mM EDTA and 0.1% Tween 20, allowed to dry, and quantified.

TABLE 2

Representative reporters and docking
domain peptides

| | | |
|---|---|---|
| SEQ ID | | |
| NO: | Name¹ | Sequence |

| | | |
|---|---|---|
| 1 | Pn-ds | PTPLSPGGGKKKPTPIQLNPAPGG |
| 2 | Pn-dsMUT | PGPLSPGGGEEEPTPAQANPAPGG |
| 3 | PnMUT-ds | PGPLSPGGGKKKPTPIQLNPAPGG |
| 4 | ds-Pn | PKKKPTPIQLNPAPGGPLSPGGGG |
| 5 | dsMUT-Pn | PEEEPTPAQANPAPGGPLSPGGGG |
| 6 | ds-PnMUT | PKKKPTPIQLNPAPGGPLAPGGGG |
| 7 | Pn(Ab1)-ds(SH2)² | EAIYAAPFAG₇GDGY(PO3)ENPSP |
| 8 | Ste7 (2-22) | FQRKTLQRRNLKGLNLNLHPD |
| 9 | scram7 (2-22) | RLQPNLQDKLNHFRTNLKGLR |
| 10 | MEK1 (1-17) | MPKKKPTPIQLNPAPDG |
| 11 | MEK2 (1-20) | MLARRKPVLPALTINPTIAE |
| 12 | MEK2_{EEAA} (1-20) | MLAEEKPVLPAATANPTIAE |
| 13 | Elk-1 (312-326) | KGRKPRDLELPLSPS |
| 14 | Elk-1_{EEG} (312-326) | KGEEPRDLEGPLSPS |
| 15 | MKP2 (71-89) | TIVRRRAKGSVSLEQILPA |
| 16 | MKP2_{EAEAA} (71-89) | TIVEAEAKGSASAEQILPA |

¹Docking domain peptides were derived from the corresponding proteins at the indicated residue positions. The peptides derive from the residue positions of the the full-length protein substrate that are indicated in parentheses.
²This reporter contains a fluorescein at the N-terminus and a phosphotyrosine at the residue denoted by “Y(PO3).”

References
Adams P D, Sellers W R, Sharma S K, Wu A D, Nalin C M, Kaelin W G Jr. “Identification of a cyclin-cdk2 recognition motif present in substrates and p21-like cyclin-dependent kinase inhibitors,” Mol. Cell. Biol. 16:6623-33, 1996.
Bardwell L, Thorner J. “A conserved motif at the amino termini of MEKs might mediate high-affinity interaction with the cognate MAPKs,” Trends Biochem. Sci. 21:373-74, 1996.
Bardwell L, Cook J G, Chang E C, Cairns B R, Thorner J. “Signaling in the yeast pheromone response pathway: specific and high-affinity interation of the mitogen-activated protein (MAP) kinases Kss1 and Fus3 with the upstream MAP kinase Kinase Ste7, ” Mol. Cell. Biol. 16:3637-50, 1996.
Bardwell A J, Flatauer L J, Matsukuma K, Thomer J, Bardwell L. “A conserved docking site in MEKs mediates high-affinity binding to MAP kinases and cooperates with a scaffold protein to enhance signal transmission,” J. Biol. Chem. 276:10374-86, 2001.
Birge R B, Hanafusa H. “Closing in on SH2 specificity,” Science 262:1522-4, 1993.
Biondi R M, Komander D, Thomas C C, Lizcano J M, Deak M, Alessi D R, van Aalten D M. “High resolution crystal structure of the human PDK1 catalytic domain defines the regulatory phosphopeptide docking site,” EMBO J. 21:4219-28, 2002.
Biondi R M, Nebreda A R. “Signalling specificity of Ser/Thr protein kinases through docking-site-mediated interaaction,” Biochem. J. 372:1-13, 2003.
Bonetta L. “Getting Proteins into Cells,” The Scientist, April:38-40, 2002.
Chen P, Hutter D, Yang X, Gorospe M, Davis R J, Liu Y. “Discordance between the binding affinity of mitogen-activated protein kinase subfamily members for MAP kinase phosphatase-2 and their ability to activate the phosphatase catalytically,” J. Biol. Chem. 276:29440-49, 2001.
Clark D E, Poteet-Smith C E, Smith J A, Lannigan D A. “Rsk2 allosterically activates estrogen receptor alpha by docking to the hormone-binding domain,” EMBO J. 20:3484-94, 2001.
Dajani R, Fraser E, Roe S M, Young N, Good V, Dale T C, Pearl L H. “Crystal structure of glycogen synthase kinase 3 beta: structural basis for phosphate-primed substrate specificity and autoinhibition,” Cell 105:721-32, 2001.
Duyster J, Baskaran R, Wang J Y. “Src homology 2 domain as a specificity determinant in the c-Abl-mediated tyrosine phosphorylation of the RNA polymerase II carboxyl-terminal repeated domain,” Proc. Natl. Acad. Sci. U.S.A. 92:1555-9, 1995.
Elia A E, Cantley L C, Yaffe M B. “Proteomic screen finds pSer/pThr-binding domain localizing Plk1 to mitotic substrates,” Science 299:1228-31, 2003.
Enslen H, Brancho D M, Davis R J. “Molecular determinants that mediate selective activation of p38 MAP kinase isoforms,” EMBO J. 19:1301-11, 2000.
Enslen H, Davis R J. “Regulation of MAP kinases by docking domains,” Biol. Cell 93:5-14, 2001.
Farooq A, Chaturvedi G, Mujtaba S, Plotnikova O, Zeng L, Dhalluin C, Ashton R, Zhou M M. “Solution structure of ERK2 binding domain of MAPK phosphatase MKP-3: structural insights into MKP-3 activation by ERK2, ” Mol. Cell 7:387-99, 2001.
Fukuda M, Gotoh Y, Nishida E. “Interation of MAP kinase witdh MAP kinase kinase: its possible role in the control of nucleocytoplasmic transport of MAP kinase,” EMBO J. 16:1901-8, 1997.
Guappone A C, Flynn D C. “The integrity of the SH3 binding motif of AFAP-110 is required to facilitate tyrosine phosphorylation by, and stable complex formation with, Src,” Mol. Cell. Biochem. 175:243-52, 1997.
Ho M S, Camiol K, Losick R. “Evidence in support of a docking model for the release of the transcription factor sigma F from the antisigma factor SpoIIAB in Bacillus subtilis,” J. Biol. Chem. 278:20898-905, 2003.
Holland P M, Cooper J A. “Protein modification: Docking site for kinases. Curr. Biol. 9:R329-R331, 1999.
Kallunki T, Deng T, Hibi M, Karin M. “c-Jun can recruit JNK to phosphorylate dimerization partners via specific docking interactions,” Cell 87:929-39, 1996.
Keyse S M. “Protein phosphatases and the regulation of mitogen-activated protein kinase signally,” Curr. Opin. Cell Biol. 12:186-192, 2000.
Kieran M W, Katz S, Vail B, Zon L I, Mayer B J. “Concentration-dependent positive and negative regulation of a MAP kinase by a MAP kinase kinase,” Oncogene 18:6647-57, 1999.
Koch C A, Moran M, Sadowski I, Pawson T. “The common src homology region 2 domain of cytoplasmic signaling proteins is a positive effector of v-fps tyrosine kinase functions,” Mol. Cell. Biol. 9:4131-40, 1989.
Leamon C P, Low PS “Folate-mediated targeting: from diagnostics to drug and gene delivery,” Drug Discovery Today, 6:44-51, 2001.
Li H, Wu H Y, et al. “Improved capillary electrophoresis conditions for the separation of kinase substrates by the laser micropipet system,” J. Chromatogr. B. Biomed. Sci. Appl. 757:79-88, 2001.
Lindgren M, Hallbrink M, Prochiantz A, Langel U. “Cell-penetrating peptides,” Trends. Pharmacol. Sci. 21:99-103, 2000.
Mayer B J, Jackson P K, Baltimore D. “The noncatalytic src homology region 2 segment of abl tyrosine kinase binds to tyrosine-phosphorylated cellular proteins with high affinity,” Proc. Natl. Acad. Sci. USA 88:627-31, 1991.
Mayer B J, Hirai H, Sakai R. “Evidence that SH2 domains promote processive phosphorylation by protein-tyrosine kinases,” Curr Biol. 5:296-305 1995.
Meredith G D, Sims C E, Soughayer J S, Allbritton N L. “Measurement of kinase activation in single mammalian cells,” Nat. Biotech. 18:309-12, 2000.
Muda M, Theodosiou A, Gillieron C, Smith A, Chabert C, Camps M, Boschert U, Rodrigues N, Davies K, Ashworth A, Arkinstall S. “The mitogen-activated protein kinase phosphatase-3 N-terminal noncatalytic region is responsible for tight substrate binding and enzymatic specificity,” J. Biol. Chem. 273:9323-29, 1998.
Neel B G, Gu H, Pao L. “The 'shp'ing news: SH2 domain-containing tyrosine phosphatases in cell signaling,” Trends Biochem. Sci. 28:284-93, 2003.
Okada C Y, Rechsteiner M. “Introduction of macromolecules into cultured mammalian cells by osmotic lysis of pinocytic vesicles,” Cell 29:33-41,1982.
Pawson T, Nash P. “Assembly of cell regulatory systems through protein interaction domains,” Science 300:445-52, 2003.
Reineke U, Volkmer-Engert R, Schneider-Mergener J. “Applications of peptide arrays prepared by the SPOT-technology,” Curr. Opin. Biotech. 12:59-64, 2001.
Ren R, Mayer B J, Cicchetti P, Baltimore D. “Identification of a ten-amino acid proline-rich SH3 binding site,” Science 259:1157-61, 1993.
Rubinfeld H, Hanoch T, Seger R. “Identification of a cytoplasmic retention sequence in ERK2, ” J. Biol. Chem. 274:30349-352, 1999.
Schalm S S, Blenis J. “Identification of a conserved motif required for mTOR signaling,” Curr. Biol. 12:632-9, 2002.

Sharrocks A D, Yang S H, Galanis A. “Docking domains and substrate-specificity determination for MAP kinases,” Trends Biochem. Sci. 25:448-53, 2000.
Sidhu S S, Bader G D, Boone C. “Functional genomics of intracellular peptide recognition domains with combinatorial biology methods,” Curr. Opin. Chem. Biol. 7:97-102, 2003.
Sims C E, Meredith G D, Krasieva T B, Bems M W, Tromberg B J, Allbritton N L. “Laser-micropipet combination for single-cell analysis,” Anal. Chem. 70:4570-7, 1998.
Slack D N, Setemes O, Gabrielsen M, Keyse S M. “Distinct binding determinants for ERK2/p38a and JNK MAP kinases mediate catalytic activation and substrate selectivity of MAP kinase phosphatase-1, ” J. Biol. Chem. 276:16491-500, 2001.
Smithgall T E. “SH2 and SH3 domains: potential targets for anti-cancer drug design,” J. Pharmacol. Toxicol. Methods. 34:125-32, 1995.
Songyang Z, Shoelson S E, Chaudhuri M, Gish G, Pawson T, Haser W G, King F, Roberts T, Ratnofsky S, Lechleider R J et al. “SH2 domains recognize specific phosphopeptide sequences,” Cell 72:767-78, 1993.
Songyang Z, Margolis B, Chaudhuri M, Shoelson S E, Cantley L C. “The phosphotyrosine interaction domain of SHC recognizes tyrosine-phosphorylated NPXY motif,” J. Biol. Chem. 270:14863-6, 1995.
Songyang Z, Lu K P, Kwon Y T, Tsai L H, Filhol O, Cochet C, Brickey D A, Soderling T R, Bartleson C, Graves D J, DeMaggio A J, Hoekstra M F, Blenis J, Hunter T, Cantley L C. “A structural basis for substrate specificities of protein Ser/Thr kinases: primary sequence preference of casein kinases I and 11, NIMA, phosphorylase kinase, calmodulin-dependent kinase 11, CDK5 and Erk1,” Mol. Cell. Biol. 16:6486-93, 1996.
Soughayer J S, Krasieva T, Jacobson S C, Ramsey J M, Tromberg B J, Allbritton N L A. “Characterization of cellular optoporation with distance,” Analytical Chemistry 72:1342-47, 2000.
Stephens D J, Pepperkok R. “The many ways to cross the plasma membrane,” Proc. Natl. Acad. Sci. USA, 98:4295-8, 2001.
Tanoue T, Adachi M, Moriguchi T, Nishida E. “A conserved docking motif in MAP kinases common to substrates, activators and regulators. Nat Cell Biol. 2:110-6, 2000.
Tanoue T, Yamamoto T, Nishida E. “Modular structure of a docking surface on MAPK phosphatases,” J. Biol. Chem. 277:22942-9, 2002.
Wang Y, Hu S, et al. “Separation of mixtures of acidic and basic peptides at neutral pH.” J. Chromatogr. A 1004 61-70, 2003.
Xia Y, Wu Z, Su B, Murray B, Karin M. “JNKK1 organizes a MAP kinase module through specific and sequential interactions with upstream and downstream components mediated by its amino-terminal extension,” Genes. Dev. 12:3369-81, 1998.
Xu B, Stippec S, Robinson F L, Cobb M H. “Hydrophobic as well as charged residues in both MEK1 and ERK2 are important for their proper docking,” J. Biol. Chem. 276:26509-15, 2001.
Yang S H, Yates P R, Whitmarsh A J, Davis R J, Sharrocks A D. “The Elk-1 ETS-domain transcription factor contains a mitogen-activated protein kinase targeting motif,” Mol. Cell. Biol. 18:710-20, 1998.
Yang S H, Galanis A, Sharrocks A D. “Targeting of p38 mitogen-activated protein kinases to MEF2 transcription factors,” Mol. Cell. Biol. 19:4028-38, 1999.
Yu J W, Lemmon M A. “Genome-wide analysis of signaling domain function,” Curr. Opin. Chem. Biol. 7:103-109, 2003.
Zheleva D I, McInnes C, Gavine A L, Zhelev N Z, Fischer P M, Lane D P. “Highly potent p21 (WAF 1)-derived peptide inhibitors of CDK-mediated pRb phosphorylation: delineation and structural insight into their interactions with cyclin A,” J. Pept. Res. 60:257-70, 2002.
Zhou B, Wu L, Shen K, Zhang J, Lawrence D S, Zhang Z Y. Multiple regions of MAP kinase phosphatase 3 are involved in its recognition and activation by ERK2. J. Biol. Chem. 276:6506-15, 2001.

1. A method of introducing a fluorescent label into a cell, comprising:
exposing a reporter to the cell,
wherein the reporter comprises:
(i) a peptide substrate for an enzyme,
(ii) a docking domain for the enzyme, attached to the peptide substrate,
(iii) a membrane traversing moiety, and
(iv) the label.

2. The method of claim 1, wherein the label is attached to the membrane traversing moiety.

3. The method of claim 1, wherein the membrane traversing moiety is a peptide transduction domain, a myristoyl moiety, or folate.

4. The method of claim 1, wherein the label is fluorescein.

5. A method of measuring the activity of a protein kinase in a cell, comprising:
introducing a membrane traversing peptide conjugate into the cell;
lysing the cell to produce a lysate;
subjecting the lysate to electrophoresis to separate a labeled reporter which reacted with the protein kinase from the labeled reporter which was unreacted;
detecting the labeled reporter; and
determining an amount of labeled reporter which reacted with the protein kinase and an amount of labeled reporter which was unreacted;
wherein the membrane traversing peptide conjugate comprises:
(i) a reporter,
(ii) a transduction domain, attached to the reporter, and
(iii) a label, attached to reporter,
wherein the reporter comprises:
(i) a peptide substrate for a protein kinase, and
(ii) a docking domain for the protein kinase, attached to the peptide substrate.

6. The method of claim 5, wherein the labeled reporter further comprises a modification, wherein the modification comprises the attachment of a moiety selected from the group consisting of phosphate moiety, a myristoyl moiety, a lipid moiety, a carbohydrate moiety, a sugar moiety, a sulfate moiety, a biotin moiety, a coordination group moiety, a ubiquitin moiety, and a nucleobase-containing moiety.

7. A reporter, comprising:
(i) a peptide substrate for an enzyme, and
(ii) a docking domain for the enzyme, attached to the peptide substrate,
wherein the enzyme is a protein kinase or phosphatase.

8. The reporter of claim 7, further comprising a label, wherein the label is attached to either the peptide substrate or the docking domain.

9. The reporter of claim 8, wherein the label is a member selected from the group consisting of a radioactive element, a fluorescent moiety, a phosphorescent moiety, a luminescent moiety, and a chemiluminescent moiety.

10. The reporter of claim 7, wherein the enzyme is a member selected from the group consisting of GMGC, STE, AGC, and TK.

11. The reporter of claim 7, further comprising a linker between the peptide substrate and the docking domain.

12. The reporter of claim 7, wherein the peptide substrate comprises a phosphoacceptor site and two flanking sequences, wherein the flanking sequences each comprise 3-8 amino acid residues.

13. A membrane traversing peptide conjugate, comprising:
(i) the reporter of claim 8, and
(ii) a transduction domain, attached to the reporter,
wherein the reporter is a substrate for a protein kinase within a cell.

14. The membrane traversing peptide conjugate of claim 13, wherein the cell is a mammalian cell.

15. The membrane traversing peptide conjugate of claim 14, wherein the mammalian cell is a human cell.

16. The membrane traversing peptide conjugate of claim 13, wherein the transduction domain is attached to the reporter by a member selected from the group consisting of a disulfide bond, a photolabile linkage and an ester linkage.

17. The membrane traversing peptide conjugate of claim 13, wherein the label is a member selected from the group consisting of a radioactive element, a fluorescent moiety, a phosphorescent moiety, a luminescent moiety, and a chemiluminescent moiety.

18. A membrane traversing peptide conjugate, comprising:
(i) the reporter of claim 8, and
(ii) a transduction domain, attached to the reporter,
wherein the reporter is a substrate for a protein kinase present in a cell,
wherein the label is fluorescent, and
wherein the cell is a mammalian cell.

19. The membrane traversing peptide conjugate of claim 18, wherein the transduction domain is attached to the reporter by a member selected from the group consisting of a disulfide bond, a photolabile linkage and an ester linkage.

20. The membrane traversing peptide conjugate of claim 19, wherein the transduction domain is attached to the reporter by a disulfide bond.

21. The membrane traversing peptide conjugate of claim 19, wherein the transduction domain is attached to the reporter by 4-[4-(1-(amino)ethyl)-2-methoxy-5-nitrophenoxy] butanoic acid.

22. The membrane traversing peptide conjugate of claim 18, wherein the label is fluorescein.

23. A membrane traversing peptide conjugate of claim 18, wherein the protein kinase is a member selected from the group consisting of GMGC, STE, AGC, and TK.

24. A method of making the membrane traversing peptide conjugate of claim 13, comprising:
synthesizing the reporter and the transduction domain;
attaching the reporter to the transduction domain; and
attaching a label to the reporter using a chemical or enzymatic means.

25. A method of introducing a labeled reporter into a cell comprising exposing the cell to the membrane traversing peptide conjugate of claim 13.

26. A method of measuring the activity of an enzyme, comprising:
exposing the reporter of claim 7 to the enzyme; and
determining the ratio of reporter which reacted with the enzyme to the reporter which was unreacted.

27. The method of claim 26, wherein the enzyme is a protein kinase.

28. A method of measuring the activity of a protein kinase in a cell, comprising:
introducing the membrane traversing peptide conjugate of claim 13 into the cell;
lysing the cell to produce a lysate;
subjecting the lysate to electrophoresis to separate the labeled reporter which reacted with the protein kinase from the labeled reporter which was unreacted;
detecting the labeled reporter; and
determining an amount of labeled reporter which reacted with the protein kinase and an amount of labeled reporter which was unreacted.

29. The reporter of claim 7, further comprising a modification to the peptide substrate, the docking domain, or both
wherein the modification comprises the attachment of a moiety selected from the group consisting of phosphate moiety, a myristoyl moiety, a lipid moiety, a carbohydrate moiety, a sugar moiety, a sulfate moiety, a biotin moiety, a coordination group moiety, a ubiquitin moiety, a nucleobase-containing moiety.

30. The reporter of claim 29, wherein the modification comprises the attachment of a phosphate moiety.

31. A membrane traversing peptide conjugate, comprising:
(i) the reporter of claim 30, and
(ii) a transduction domain, attached to the reporter,
wherein the reporter is a substrate for a protein kinase within a cell.

32. A membrane traversing peptide conjugate, comprising:
(i) the reporter of claim 30, and
(ii) a transduction domain, attached to the reporter,
wherein the reporter is a substrate for a protein phosphatase within a cell.

33. The membrane traversing peptide conjugate of claim 31, further comprising a label, wherein the label is attached to the reporter.

34. A method of introducing a labeled reporter into a cell comprising exposing the cell to the membrane traversing peptide conjugate of claim 33.

35. The reporter of claim 30, further comprising a label, wherein the label is attached to the peptide substrate.

36. A method of measuring the activity of a protein kinase, comprising:
exposing the reporter of claim 35 to the protein kinase; and
determining the ratio of reporter which reacted with the protein kinase to the reporter which was unreacted.

37. A reporter, comprising:
(i) a substrate for an enzyme, and
(ii) a docking domain for the enzyme, attached to the substrate.

38. The reporter of claim 37, further comprising a label, wherein the label is attached to either the substrate or the docking domain.

39. The reporter of claim 38, wherein the label is a member selected from the group consisting of a radioactive element, a fluorescent moiety, a phosphorescent moiety, a luminescent moiety, and a chemiluminescent moiety.

40. The reporter of claim 39, further comprising a modification to the substrate, the docking domain, or both,
wherein the modification comprises the attachment of a moiety selected from the group consisting of phosphate moiety, a myristoyl moiety, a lipid moiety, a carbohydrate moiety, a sugar moiety, a sulfate moiety, a biotin moiety, a coordination group moiety, a ubiquitin moiety, and a nucleobase-containing moiety.

41. A method of measuring the activity of an enzyme, comprising:
exposing the reporter of claim 39 to the enzyme; and
determining the ratio of reporter which reacted with the enzyme to the reporter which was unreacted.
